(19) United States
(12) Patent Application Publication
(10) Pub. No.: US 2019/0154852 A1
(43) Pub. Date: May 23, 2019

(54) Analog Direct Digital X-Ray Photon Counting Detector For Resolving Photon Energy In Spectral X-Ray CT

(71) Applicant: William McCroskey, Rootstown, OH (US)

(72) Inventor: William McCroskey, Rootstown, OH (US)

(73) Assignee: NueVue Solutions, Inc., Rootstown, OH (US)

(21) Appl. No.: 16/193,550

(22) Filed: Nov. 16, 2018

Related U.S. Application Data
(60) Provisional application No. 62/586,969, filed on Nov. 16, 2017.

(51) Int. Cl. G01T 1/24; A61B 6/00; A61B 6/03; G01T 1/29; G01T 1/36

(52) U.S. Cl. CPC G01T 1/247; A61B 6/585; A61B 6/032; G01T 1/366; G01T 1/249; G01T 1/2985; A61B 6/5205

(57) ABSTRACT

An analog x-ray photon counting detector is provided. The detector may include a direct conversion medium such as CZT, a charge sensitive preamplifier receiving an electronic pulse form the direct conversion medium, pulse-shaping electronics for conditioning the amplified signal, and one or more time-over-threshold triggers set to differing trigger levels. The time-over-threshold data is the related back to photon energy through a calibration curve, where each trigger level is associated with one calibration curve. The calibration data may be contained in a nonlinear lookup table. Each photocurrent pulse may be analyzed according to one or more time-over-threshold measurements. Thus, the energy values computed from each-time-over threshold measurement may be averaged.

### I. BACKGROUND OF THE INVENTION

### A. Field of Invention

The invention generally relates to the field of therapeutic-diagnostic (theranostic) molecular imaging using spectral CT.

### B. Description of the Related Art

Simple two-dimensional x-ray images have long been made using x-ray projection radiography devices. This type of instrument often includes an x-ray source and x-ray sensitive photographic film, although electronic imaging is also known in x-ray projection radiography. When an arm or other body part of a patient is placed between the x-ray source and film, a portion of the x-ray photons are absorbed by the tissues, bone, and other biological materials, a portion is scattered, and a portion is transmitted to the film to form the image. The film does not discriminate between different wavelengths of x-ray photons, so the image produced is essentially the difference between the number of photons incident on the patient versus the number of photons transmitted to the film, distributed over a two-dimensional field of view. The difference image is therefore monochromatic.

Similarly, traditional x-ray CT instruments also collect x-ray attenuation data, but rather than photographic film, a semi-circular array of x-ray cameras are positioned about the patient. The cameras often comprise arrays of scintillation crystals. Accordingly, a transmitted x-ray photon impinges a scintillation crystal where it is absorbed and emits a proportional number of lower-energy photons. These in turn are read by photomultiplier tubes. The photomultiplier tubes convert light pulses from the scintillation crystal to analog electrical pulses which are then digitized by an analog-to-digital converter. The digital signal is further processed and a three-dimensional image is reconstructed. Although x-ray CT uses semicircular camera arrays rather than film, it still collects monochromatic images. It is known to introduce contrast agents into patient tissues which have a larger x-ray capture cross section than the surrounding tissue. This practice enhances an instrument's ability to distinguish soft tissues which have relatively low x-ray attenuation, but the image is still a monochromatic difference image.

Spectral CT takes advantage of the fact that most conventional x-ray sources, such as the widely-used rotating anode source, are inherently polychromatic. Spectral CT instruments use photon counting electronics to determine the wavelength of each x-ray photon. However, the electrical pulses produced still must be digitized through an analog-to-digital converter which takes time and can result in information loss at higher photon fluxes.

What is missing in the art is hardware and methodology for collecting photon counting data without the need for an analog to digital converter. Some embodiments of the present invention may provide such hardware an methodology.

### II. SUMMARY OF THE INVENTION

Some embodiments may relate to an analog x-ray photon counting detector. The detector may include a direct conversion medium electronically responsive to x-ray and/or gamma photons such that the direct conversion medium generates an analytically useful photoelectronic pulse proportional to an energy of an absorbed photon. The detector may further include a charge sensitive preamplifier in electronic communication with the direct conversion medium and receptive to the photoelectronic pulse as input, wherein the charge sensitive preamplifier outputs an electronically useful pulse proportional to the photoelectronic pulse input. The detector may further include a pulse-shaping amplifier receptive to the output of the charge sensitive preamplifier as input and produces an analytical signal pulse. The detector may further include a first electronic counter-timer in electronic controlling communication with a first AND gate such that the first electronic counter-timer starts when triggered at a first trigger level in a rise time of the analytical signal pulse and the first electronic counter-timer stops when triggered at the first trigger level in a fall time of the analytical signal pulse. The detector may further include a second electronic counter-timer in electronic controlling communication with a second AND gate such that the second electronic counter-timer starts when triggered at a second trigger level in the rise time of the analytical signal pulse and the second electronic counter-timer stops when triggered at the second trigger level in the fall time of the analytical signal pulse. The detector may further include a processor suitably programmed to compare the outputs of the first and second first electronic counter-timers to a look up table of calibration data relating said outputs to photon energy.

Other benefits and advantages will become apparent to those skilled in the art to which it pertains upon reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, wherein like reference numerals indicate like structure, and wherein:

FIG. 1 is a generalized energy spectrum of a typical rotating anode x-ray source;

FIG. 2 is a plot of linear attenuation coefficients of several theranostically important materials within the normal operating range of a typical rotating anode source;

FIG. 3 is a plan view of a 32×32 grid of CZT direct conversion crystals;

FIG. 4 is a perspective view of CZT crystal bonded to a 1024 channel interposer board;

FIG. 5A shows the interposer board of FIG. 4 in relation to an FPGA board;

FIG. 5B shows a stack of FPGA boards;

FIG. 6 is a circuit diagram selected electronics of an embodiment;

FIG. 7A shows a Δt measurement at a first trigger level;

FIG. 7B shows a Δt measurement at a second trigger level;

FIG. 7C shows a Δt measurement at a third trigger level; and

FIG. 8 is a plot of three calibration curves of a three-trigger level embodiment.

### IV. DETAILED DESCRIPTION OF THE INVENTION

As used herein the terms “embodiment”, “embodiments”, “some embodiments”, “other embodiments” and so on are not exclusive of one another. Except where there is an explicit statement to the contrary, all descriptions of the features and elements of the various embodiments disclosed herein may be combined in all operable combinations thereof.

Language used herein to describe process steps may include words such as “then” which suggest an order of operations; however, one skilled in the art will appreciate that the use of such terms is often a matter of convenience and does not necessarily limit the process being described to a particular order of steps.

Conjunctions and combinations of conjunctions (e.g. “and/or”) are used herein when reciting elements and characteristics of embodiments; however, unless specifically stated to the contrary or required by context, “and”, “or” and “and/or” are interchangeable and do not necessarily require every element of a list or only one element of a list to the exclusion of others.

Terms of degree, terms of approximation, and/or subjective terms may be used herein to describe certain features or elements of the invention. In each case sufficient disclosure is provided to inform the person having ordinary skill in the art in accordance with the written description requirement and the definiteness requirement of 35 U.S.C. 112.

The invention relates to spectral CT detector electronics and methods for resolving photon energy in a single photon counting detection regime. The electronics and methods provided eliminate per-pixel analog-to-digital conversion hardware. Moreover, embodiments of the invention may increase the speed of the detector, add multiple energy bins, and decrease detector deadtime. Embodiments may advantageously include direct conversion materials for x-ray photon detection, and may further include an empirical multi-trigger level time-over-threshold method for resolving photon energy.

Direct conversion detector materials within the scope of the invention include, without limitation, one or more of CdZnTe (CZT), CdTe, amorphous selenium, GaAs, HgI₂, PbO, PbI₂, and/or methyl ammonium lead triiodide perovskite (MAPbI₃). The person having ordinary skill in the art will understand that direct conversion is a term of art referring to semiconductor crystals that convert gamma photons directly into electrons. This is in contrast to scintillation crystals which convert gamma photons into lower-energy photons. The lower-energy photons are in turn detected by photomultiplier tubes or similar electronics, e.g. charge coupled devices (CCDs). In a direct conversion crystal like CZT electrical contacts are bonded to the surface of each crystal element, i.e. pixel, to form a cathode and an anode through which an electric field is applied to the crystal element. Accordingly, when the crystal absorbs a gamma photon, an electron-hole pair is created which separately move through the field to the corresponding electrode, thereby creating a photocurrent.

The skilled artisan will appreciate that any direct conversion material may be appropriate provided that it is capable of producing an analytically useful photoelectronic pulse. In this context the term “analytically useful” means that the photoelectronic pulse is proportional to the energy of an absorbed photon, or is otherwise mathematically relatable to the energy of the absorbed photon such that photon energy information can be recovered from the photoelectronic pulse. More specifically, the number of electrons excited to the conduction band of the direct conversion material is proportional, or otherwise mathematically relatable, to the energy of the absorbed photon. Analytically useful further means that the photoelectronic pulse is capable of being received and amplified by a preamplifier for further processing to recover photon energy information.

Suitable preamplifiers for receiving and amplifying the photoelectronic pulse include charge sensitive preamplifiers. Charge sensitive preamplifiers may be particularly advantageous due to their inherent capacity to produce output signals that are charge-proportional to their input signals. The skilled artisan will appreciate that this is advantageous but not required for relating the preamplifier output signal back to photon energy. The output of the preamplifier is electronically useful, meaning that it is capable of yielding meaningful data because it has, for instance and without limitation, an amplitude, signal strength, and/or signal-to-noise ratio sufficient for further processing by downstream electronics.

Embodiments may also include a pulse shaping component(s) that receives the output of the preamplifier and further conditions the signal to convert it to a form suitable for making time-over-threshold measurements. Suitable shaping components may perform operations such as baseline correction and/or may produce a very fast rectangular-shaped gaussian pulse. The person having ordinary skill in the art will readily appreciate a variety of structures and arrangements for performing such operations, all of which are within the scope of the present invention. One suitable structure is a pulse shaping preamplifier. The output signal of the pulse shaping component(s) is referred to herein as the analytical signal.

Embodiments may communicate the analytical signal to a plurality of triggers set to predetermined trigger levels. While the specific value to which each trigger is set is non-critical the levels are selected for the purpose of collecting pulse shape information so that the shape of the analytical signal pulse may be estimated or inferred. Each trigger level may be AND-gated to a counter-timer so that the timer switches between on and off states whenever the trigger level is passed. Accordingly, when a given trigger level is passed during the rise time of an analytical signal pulse the associated AND-gate turns on the electronic counter-timer and when the trigger level is passed during the decay time of the same pulse the counter-timer is switched back to the off state. Thus, the value of the counter-timer may be read to obtain a time-over-threshold measurement of the pulse.

Likewise, one or more other counter-timers may be similarly AND-gated to triggers set to different trigger levels that may be sufficiently spaced apart to provide an estimate of peak height and width. The person having ordinary skill in the art will appreciate that any number time-over-threshold measurements may be similarly obtained at various trigger levels. Advantageously, time-over-threshold measurements should be taken at trigger levels sufficiently spaced apart to minimize the number of triggers while still obtaining useful data. In this context the term useful data means data that are suitable for accurately calculating photon energy through comparison to one or more calibration curves.

Some embodiments may comprise one or more calibration curves. Each calibration curve corresponds to a trigger level and may comprise a plot of photon energy versus time over threshold (Δt). According to embodiments of the invention, photon energy increases as a nonlinear function of time over threshold. Thus, for a give trigger level a calibration curve can be constructed relating time over threshold Δt to photon energy. Such curves can be constructed using radio isotope standards with well-known monochromatic emissions.

With respect to using the calibration curves, in theory, a plurality of triggers analyzing the same photocurrent peak should measure time over threshold values that relate back to the same photon energy. In practice a plurality of triggers are used and the resulting energies are averaged.

Such calibration curves may be recorded in, for example and without limitation, a non-linear lookup table data structure. The skilled artisan will appreciate that there are many ways in which calibration data may be stored, structured, and queried for making comparisons. All such variations are intended to be within the scope of the present invention. Likewise, the skilled artisan will readily appreciate that there are many known mathematical methods for calculating photon energy from calibration curves. One well-known method is the least squares fit; however, any suitable fitting algorithm is also within the scope of the present invention.

After successfully relating an analytical pulse to a photon energy the pulse may be assigned to one or more energy bins. Each pixel may have a plurality of energy bins corresponding in number to the number of different photon energies among which the pixel may discriminate. Each energy bin may represent a predetermined photon energy and may comprise an accumulator adapted to accumulate counts relatable to the number of analytical pulses assigned to the bin. Bin data may be read and used to construct histograms for image reconstruction according to any of a wide variety of well-known methods. Thus, each pixel may have associated with it a plurality of histograms comprising measurements of intensity, or photon flux, at each photon energy during an accumulation period.

Depending on context, the term pixel may mean an element of the direct conversion medium and/or any part of, or all of, the downstream electronic components for processing photonic data collected by the element of the direct conversion medium, including determining photon energy and photon counts for image reconstruction.

Having the capacity to discriminate among a number of different photon energies permits embodiments of the present invention to discern structures that would otherwise be invisible in a monochromatic attenuation image. For instance, soft tissue structures become more readily discernable from bone, and a plurality of metals can be simultaneously distinguished in a single image. Accordingly, suitably functionalized nanoparticle formulations may be administered to a patient and imaged.

Functionalized nanoparticles may be particularly advantageous where they are administered to a localized area such as a joint, or where they are functionalized with materials such as antibodies that are specific to a physiological structure of interest, such as a tumor, or a biofilm-forming bacterial infection on a titanium implant or other device implanted in the bone. Having the capacity to image physiological structures within the body provides the physician with very specific position information that may be suitable for automatically directing medical instruments. For example, and without limitation, a plurality of pulsed infrared lasers may be placed around, and registered to, a biofilm structure such that the beams cross at a common target point of the biofilm. Then ablation pulses may be administered according to a suitable pulse sequence to kill the bacterial biofilm without destroying the surrounding healthy tissue.

The electronics and methods provided by the present invention are also useful for other forms of medical imaging including, without limitation, positron emission tomography (PET) and single-photon emission computed tomography (SPECT). The person having ordinary skill in the art will readily understand that PET and SPECT imaging technologies both detect gamma photons using either scintillation crystals or direct conversion media such as CZT. Moreover, the inherently low photon fluxes sensed by PET and SPECT instruments lend themselves to photon counting. Accordingly, the present invention provides ultra-fast detection electronics suitable not only for the spectral CT methods described herein, but also PET and SPECT methods.

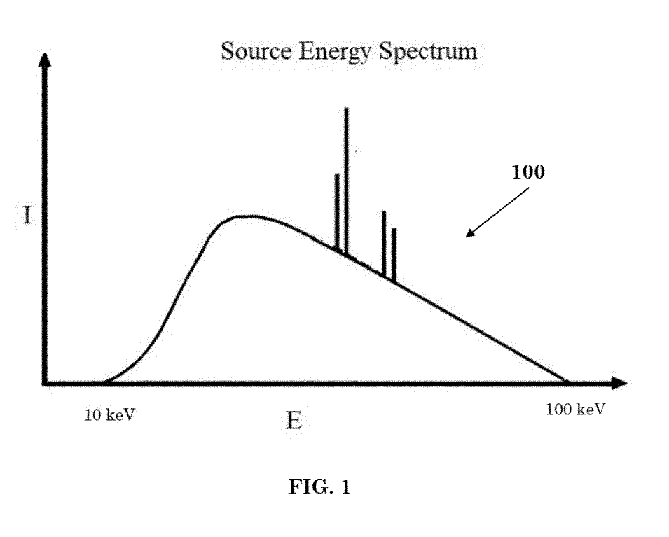

Referring now to the drawings wherein the showings are for purposes of illustrating embodiments of the invention only and not for purposes of limiting the same, FIG. 1 is a generalized energy spectrum 100 of a typical rotating anode x-ray source. Such sources are inherently polychromatic over a broad range of photon energies due to electron deceleration as they approach the anode target, which is referred to as Bremsstrahlung radiation. The sharp peaks within the energy spectrum are the K_{α} and K_{β} emissions as well as other emissions due to ejection of core electrons.

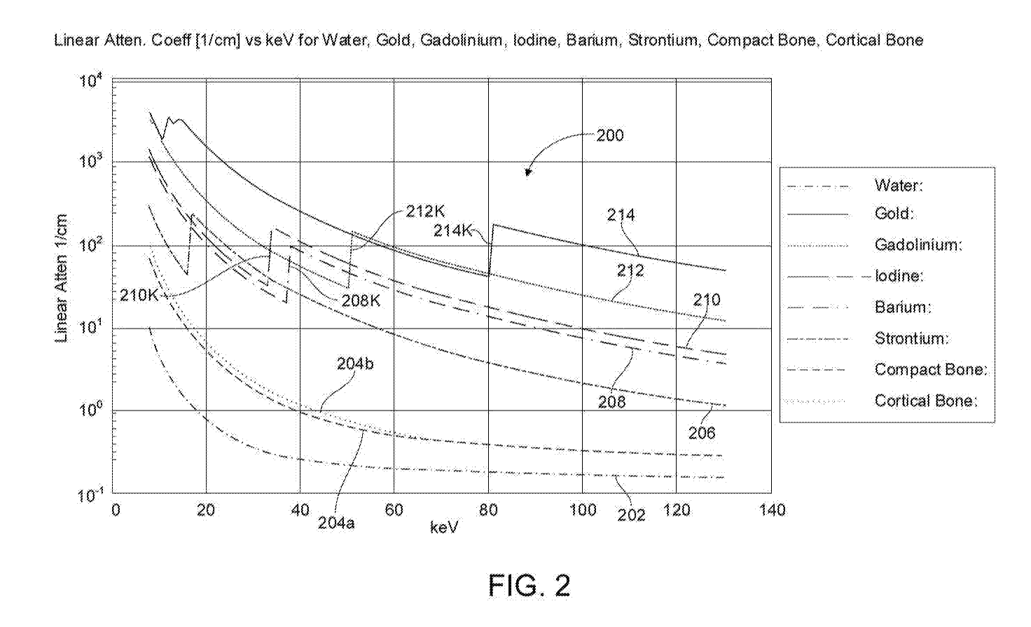

FIG. 2 illustrates the attenuation curves 200 of various materials such as water 202, compressed bone 204a, cortical bone 204b, strontium 206, barium 208, iodine 210, gadolinium 212, and gold 214. As shown in FIG. 2, physiological materials like bone 204a, 204b and water 202 exhibit very low attenuation due to the relatively small capture cross sections of their constituent atoms which is a function of their relatively low atomic numbers. In contrast, higher atomic number materials such as gold 214 have commensurately higher attenuations. Advantageously, higher atomic number materials such as gold 214, exhibit K edge absorptions 214K within an analytically useful part of the energy spectrum of typical x-ray sources. The person having ordinary skill in the art will understand what is meant by analytically useful in this context to be that the spectral output of the source is sufficiently high to allow for suitable signal-to-noise ratios. This is particularly evident by comparing FIG. 1 and FIG. 2 which both show spectral energies between 10 and 100 KeV. Thus, a mixture of gold 214, gadolinium 212, barium 208, and iodine 210 may be administered simultaneously, imaged, and distinguished by their signature K edge absorptions, i.e. 214K, 212K, 208K and 210K respectively.

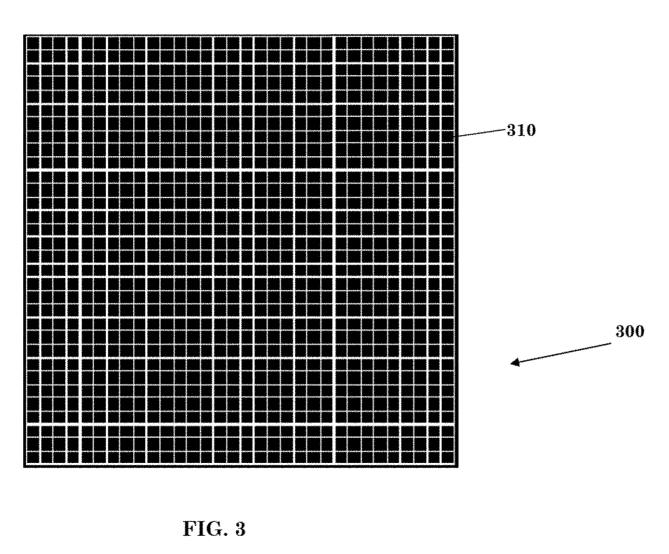

FIG. 3 is a plan view illustration of a 32×32 array of CZT crystals 300 comprising 1024 direct conversion crystal elements 310. The person having ordinary skill in the art will understand that each CZT crystal includes an anode and a cathode bonded thereto, which are not shown.

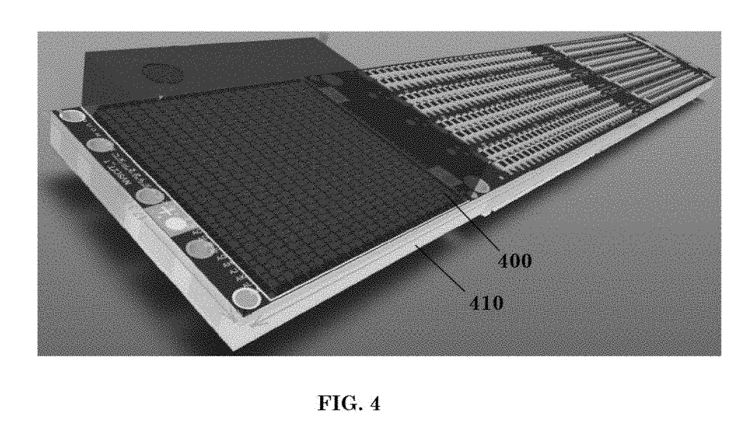

FIG. 4 illustrates a CZT crystal 400 or crystal array 300 bonded to a 1024 channel interposer board 410. The interposer board 410 may optionally contain the electric charge pulse amplifiers discussed herein (e.g. charge sensitive preamplifiers); however, the skilled artisan will readily appreciate that this is not a requirement. Amplifying components may be located elsewhere as a matter of design choice.

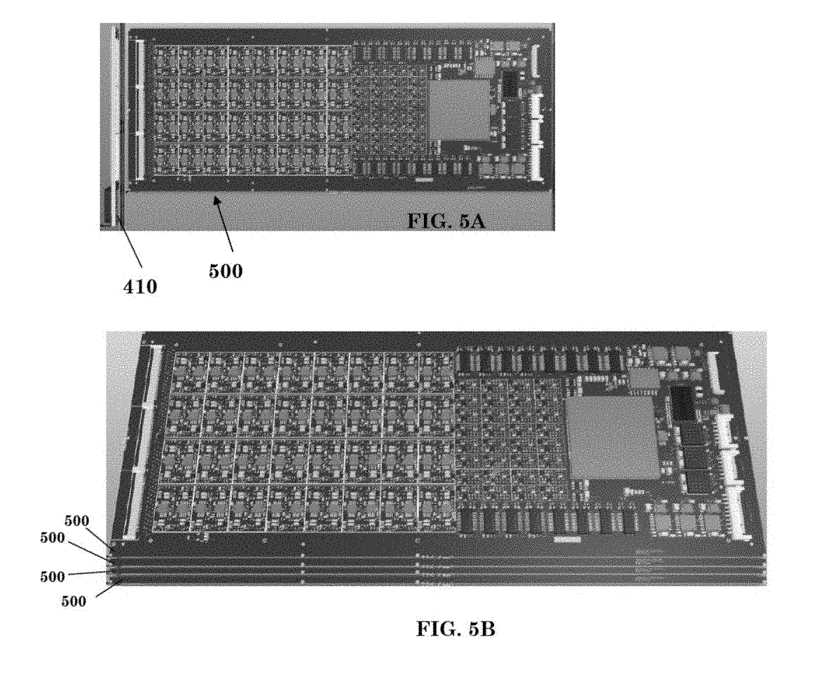

A general-purpose FPGA board 500 is shown in FIG. 5A to which the interposer board 410 of FIG. 4 connects and communicates pulse data for processing. While this figure illustrates a commercially available general-purpose FPGA board it will be readily understood by the person having ordinary skill in the art, that a purpose-built FPGA board may also be used, and may include some or all of the components illustrated on the interposer board 410.

FIG. 5B illustrates that a stack of four or more FPGA boards 500 may be combined in a multi-detector array. The person having ordinary skill in the art will appreciate that the functionality of the interposer 410 and FPGA 500 may be integrated into a single board, or even a single integrated circuit.

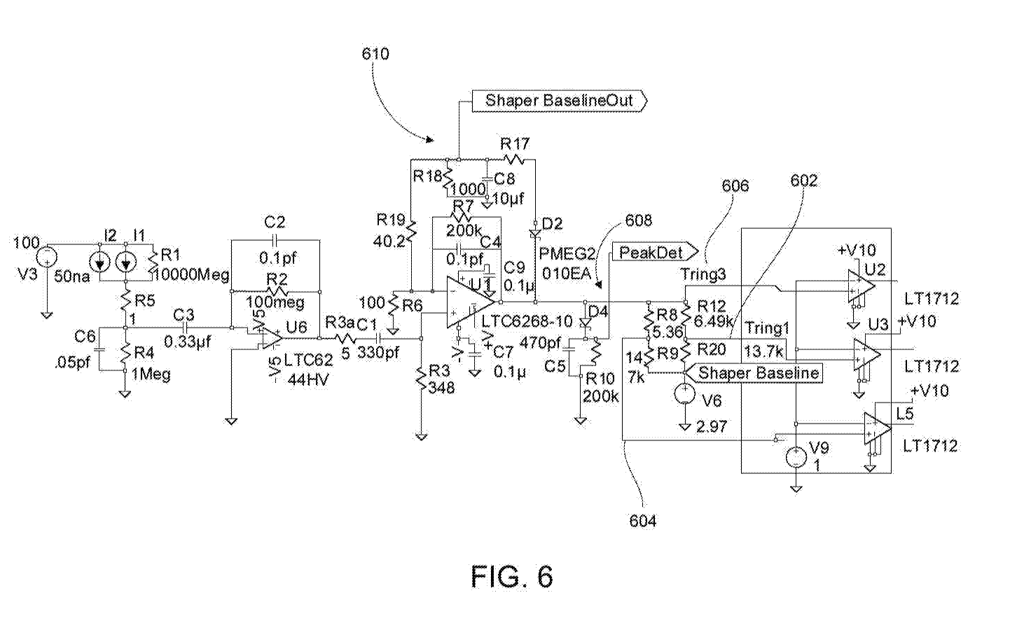

FIG. 6 illustrates a circuit of the present invention including charge sensitive preamplifiers, and pulse shaping and baseline correction components as well as programmable trigger electronics. For example, according but not limited to the embodiment shown in FIG. 6, the circuit includes a first trigger 602, a second trigger 604, and a third trigger 606, an average peak detector level 608, and a baseline restore adjustable for count rate 610. With the benefit of this circuit diagram, the person having ordinary skill in the art will readily understand that other components and arrangements of components may have the same or equivalent functionality, and thus are within the scope of the invention.

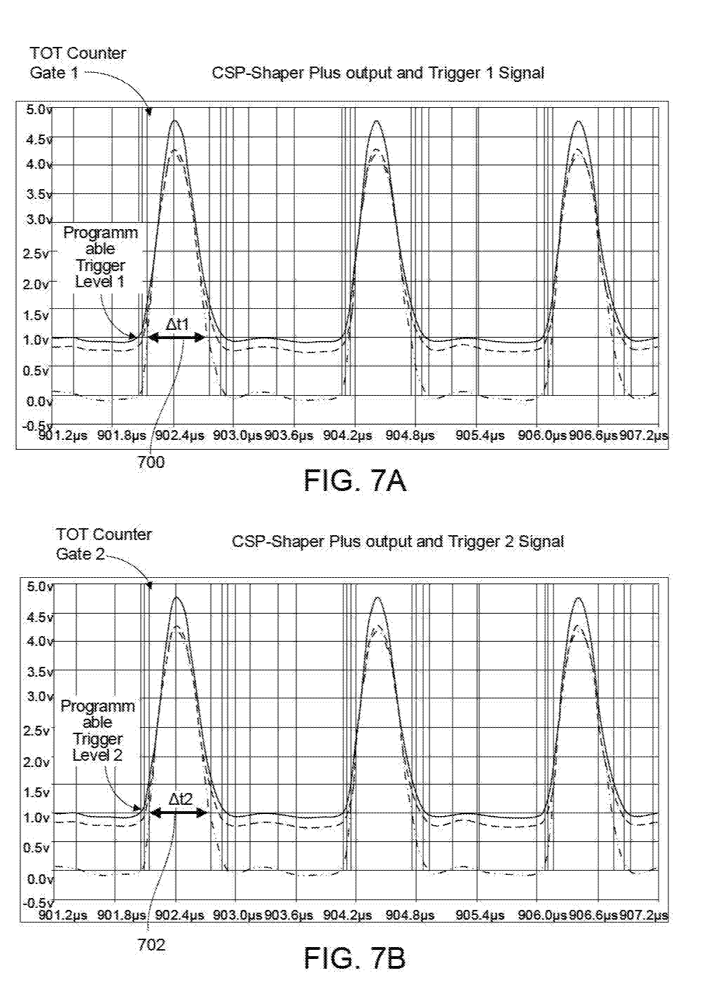

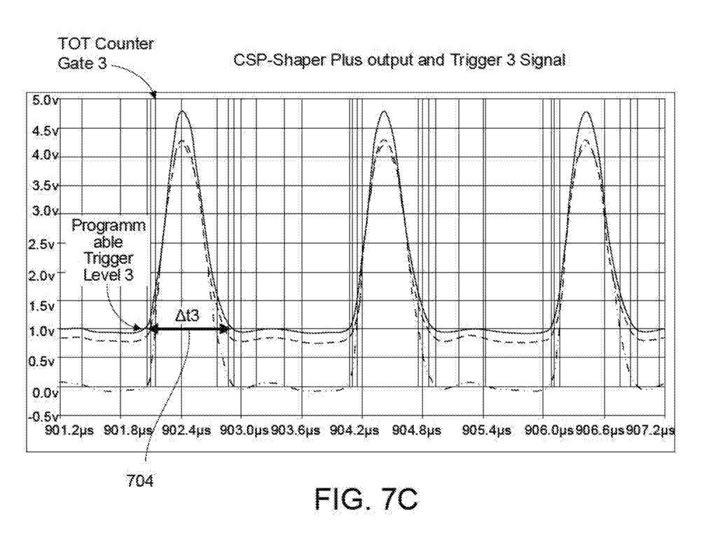

The skilled artisan will readily appreciate that some off-the-self FPGA hardware has a fixed trigger level, while others may have programmable trigger levels. The example data shown in FIGS. 7A-7C illustrate an embodiment including an FPGA having a fixed trigger level. In order to obtain time-over-threshold measurements at different levels for each photon detection event the photon signal is split into three replicates where each is scaled by adding a constant off-set value. For instance, one retains a baseline of 0.00V which is advantageously set just above the noise level, a second is shifted positively by adding a constant 0.75V, and a third is shifted positively by adding a constant 1.00V. Thus, three different triggers, all set to the same level (e.g. 1.00V), can be used to collect data over a single photon peak by shifting the peak rather than the trigger level. The skilled artisan will appreciate that this is equivalent to setting three different triggers at 1.00V, 0.75V, and 0.00V.

With continuing reference to FIGS. 7A-7C, the photonic data has been amplified, pulse-shaped, and baseline corrected. Each set of three overlaid peaks centered a common time represents a single photon detection event, and is suitable for time-over-threshold measurements. Three different counter timers are AND-gated to each of three triggers arbitrarily set to about 1.00V. The counter timers collect three different Δt values between their on and off states. The length of lines 700, 702, and 704 corresponds to the values of Δt₁, Δt₂, and Δt₃. These measurements may be collectively compared to empirical calibration data contained in a non-linear look-up table to calculate a photon energy for each pulse according to a least squares method.

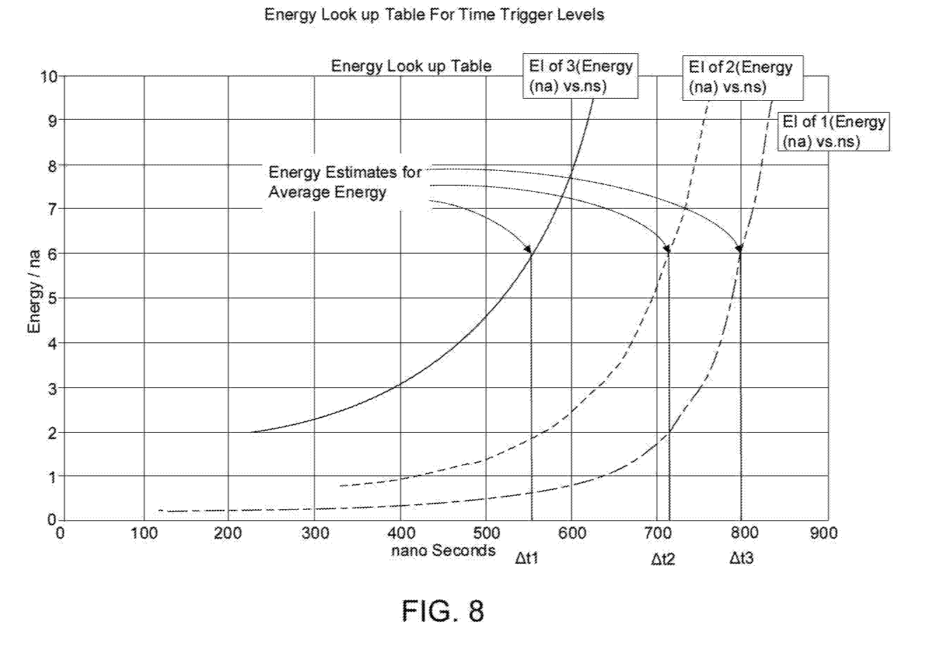

Sample calibration data is shown in FIG. 8 which is shown in the form of three calibration curves, corresponding to the three triggers, for the sake of illustration. Time in nanoseconds is shown in the x-axis and energy is shown on the y-axis. Each of Δt₁, Δt₂, and Δt₃ correspond to the same photon energy value. Since each Δt is a measurement of the same photon detection event, each Δt must correspond to the same energy value, namely the energy of the detected photon. The skilled artisan will appreciate that the Δt values may not perfectly correspond, owing to the noise level inherent in any physical measurement. Thus, the energy values corresponding to each Δt may be averaged, and a standard deviation may be calculated to establish the quality of the measurement.

Suitable means for calibration of spectral CT instruments are well known in the art, all of which are within the scope of the invention. One method uses the monochromatic nature of photons emitted from radioisotopes which are emitted at well-known energies thus serving as suitable photon energy standards. The typical dynamic range of a spectral CT instrument is between about 10 keV and 130 keV. Thus, suitable photon standards have energies within that range. According to one calibration method of the invention, decay of americium-241 (²⁴¹Am) emits a photon at 59.54 keV.

It will be apparent to those skilled in the art that the above methods and apparatuses may be changed or modified without departing from the general scope of the invention. The invention is intended to include all such modifications and alterations insofar as they come within the scope of the appended claims or the equivalents thereof.

Having thus described the invention, it is now claimed:

I claim:

1. An analog x-ray photon counting detector, comprising:
a direct conversion medium electronically responsive to x-ray and/or gamma photons such that the direct conversion medium generates an analytically useful photoelectronic pulse proportional to an energy of an absorbed photon;
a charge sensitive preamplifier in electronic communication with the direct conversion medium and receptive to the photoelectronic pulse as input, wherein the charge sensitive preamplifier outputs an electronically useful pulse proportional to the photoelectronic pulse input;
a pulse-shaping amplifier receptive to the output of the charge sensitive preamplifier as input and produces an analytical signal pulse;
a first electronic counter-timer in electronic controlling communication with a first AND gate such that the first electronic counter-timer starts when triggered at a first trigger level in a rise time of the analytical signal pulse and the first electronic counter-timer stops when triggered at the first trigger level in a fall time of the analytical signal pulse;
a second electronic counter-timer in electronic controlling communication with a second AND gate such that the second electronic counter-timer starts when triggered at a second trigger level in the rise time of the analytical signal pulse and the second electronic counter-timer stops when triggered at the second trigger level in the fall time of the analytical signal pulse; and
a processor suitably programmed to compare the outputs of the first and second first electronic counter-timers to a look up table of calibration data relating said outputs to photon energy.

2. The analog x-ray photon counting detector of claim 1 further comprising a third electronic counter-timer in electronic controlling communication with a third AND gate such that the third electronic counter-timer starts when triggered at a third trigger level in the rise time of the analytical signal pulse and the third electronic counter-timer stops when triggered at the third trigger level in the fall time of the analytical signal pulse, wherein the processor is further programmed to compare the output of the third electronic counter-timer, in addition to the outputs of the first and second electronic counter-timers, to the look up table of calibration data relating said outputs to photon energy.

3. The analog x-ray photon counting detector of claim 1, wherein the direct conversion medium comprises one or more of CZT, CdTe, amorphous selenium, GaAs, HgI₂, PbO, PbI₂, and/or methyl ammonium lead triiodide perovskite (MAPbI₃).

4. The analog x-ray photon counting detector of claim 1, wherein the analytical signal pulse output by the pulse-shaping amplifier is a count rate baseline corrected pulse.

5. The analog x-ray photon counting detector of claim 1, wherein the direct conversion medium comprises one pixel of an array of substantially identical pixels.

6. The analog x-ray photon counting detector of claim 5, wherein the analytical signal pulse comprises the sum of substantially simultaneous electrical outputs of the pixel and of each adjacent pixel surrounding the pixel.

7. The analog x-ray photon counting detector of claim 5, wherein the analytical signal pulse comprises, directly or indirectly, a sum of substantially simultaneous electrical outputs of discrete adjacent members of the direct conversion medium consisting of a central member and its nearest neighbor members.

8. The analog x-ray photon counting detector of claim 1, wherein the processor is suitably programmed to derive energy, time, x-y, position, angular rotation position, and physiological signals from the analytical signal pulse and record the derived energy, time, x-y, position, angular rotation position, and physiological signals in a digital format.

9. The analog x-ray photon counting detector of claim 1, further comprising a plurality of accumulators corresponding to a pixel, the accumulators each comprising an energy bin corresponding to a predetermined photon energy of interest, wherein the photon energy is recorded as a count in the energy bin corresponding to the photon energy.

10. The analog x-ray photon counting detector of claim 9, wherein the processor is suitably programmed to compute energy histograms from count data recorded in the energy bins.
